(19) United States
(12) Patent Application Publication
(10) Pub. No.: US 2004/0161412 A1
(43) Pub. Date: Aug. 19, 2004

(54) Cell-based VEGF delivery

(75) Inventors: Marc S. Penn, Shaker Heights, OH (US); Arman T. Askari, University Heights, OH (US); Matthew Kiedrowski, Lorain, OH (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: The Cleveland Clinic Foundation

(21) Appl. No.: 10/426,769

(22) Filed: Oct. 1, 2003

Related U.S. Application Data
(60) Provisional application No. 60/405,274, filed on Aug. 22, 2002.
(60) Provisional application No. 60/424,065, filed on Nov. 6, 2002.

(51) Int. Cl. A61K 45/00; A61K 38/18

(52) U.S. Cl. 424/93.7; 514/12

(57) ABSTRACT

A method of stimulating stem cell differentiation in ischemia damaged tissue comprises the steps of increasing the concentration of VEGF in the ischemia damaged tissue and increasing the concentration of stem cells in the ischemia damaged tissue while the concentration of VEGF in the ischemia damaged tissue is increased.

### FIELD OF THE INVENTION

The present invention relates to a method of stimulating stem cell differentiation and particularly relates to a method of stimulating stem cell differentiation using cell-based VEGF delivery.

### BACKGROUND OF THE INVENTION

Therapeutic angiogenesis offers the potential for treatment of ischemic cardiomyopathy and other ischemic syndromes. Therapeutic angiogenesis for the treatment of ischemic heart disease has demonstrated efficacy in several animal models and human pilot trials in patients not suitable for coronary revascularization. Angiogenic factors tested in human trials include FGF-1,-2 and -4 and several multiple isoforms of VEGF. Freedman, S. B. & Isner, J. M., Ann Intern Med 2002;

### FIELD OF THE INVENTION

The present invention relates to a method of stimulating stem cell differentiation and particularly relates to a method of stimulating stem cell differentiation using cell-based VEGF delivery.

### BACKGROUND OF THE INVENTION

Therapeutic angiogenesis offers the potential for treatment of ischemic cardiomyopathy and other ischemic syndromes. Therapeutic angiogenesis for the treatment of ischemic heart disease has demonstrated efficacy in several animal models and human pilot trials in patients not suitable for coronary revascularization. Angiogenic factors tested in human trials include FGF-1,-2 and -4 and several multiple isoforms of VEGF. Freedman, S. B. & Isner, J. M., Ann Intern Med 2002; 136(1):54-71. The optimal delivery method for angiogenesis treatment has yet to be determined. Persistent and unregulated vascular endothelial growth factor (VEGF) production has untoward affects in animal models, therefore, local and transient expression, in an attempt to minimize systemic effects is preferred. Lee et al., Circulation 2000; 102(8):898-901.

Strategies studied in clinical populations for delivery of angiogenic factors have included delivery of protein through intravenous or intracoronary injection, intracoronary injection of adenovirus, or direct intramyocardial injection of protein, naked DNA or adenovirus encoding for angiogenic gene products. Udelson, J. E., et al., Circulation 2000; 102(14):1605-1610. Simons, M., et al., Chronos N A. Circulation 2002; 105(7):788-793. Grines, C. L., et al. Circulation 2002; 105(11):1291-1297. Laham, R. J., et al., Circulation 1999; 100(18):1865-1871. Vale, P. R., et al., Circulation 2001; 103(17):2138-2143. Rosengart T. K., et al. Circulation 1999; 100(5):468-474.

Intracoronary delivery strategies of VEGF protein are limited by systemic toxicities including hypotension that develop in response to the high doses required to obtain sufficient myocardial revascularization. Hariawala M. D., et al., J. Surg. Res. 1996; 63(1):77-82. Lopez, J. J., et al. Am. J. Physiol. 1997; 273(3 Pt 2):H1317-H1323. Naked DNA requires direct myocardial injection due to rapid degradation by circulating nucleases.

### SUMMARY OF THE INVENTION

One aspect of the present invention relates to a method of stimulating stem cell differentiation in ischemia damaged tissue. The ischemia damaged tissue includes a first concentration of stem cells and a first concentration of VEGF. In the method, the concentration of VEGF in the ischemia damaged tissue can be increased from a first concentration to a second concentration. The concentration of stem cells in the ischemia damaged tissue can be increased from the first concentration to a second concentration. The concentration of stem cells can be increased while concentration of VEGF in the ischemia damaged tissue is increased.

In accordance with another aspect of the present invention, the number of stem cells can be increased by either administering an agent that causes stem cells to mobilize from bone marrow to the peripheral blood of the ischemia damaged tissue or injecting stem cells into the peripheral blood. In a preferred aspect of the present invention, the agent that causes the stem cells to mobilize from the bone marrow to the peripheral blood can be selected from the group consisting of cytokines, chemokines, and chemotherapeutic agents. In a more preferred aspect of the present invention, the agent comprises granulocyte colony stimulating factor (G-CSF).

In another aspect of the present invention the step of increasing the concentration of VEGF comprises affecting cells to express VEGF in the ischemia damaged tissue. The cell can be affected to express VEGF using gene therapy. A preferred method of gene therapy can include transfecting the cells with an expression vector. The expression vector can include a nucleic acid encoding VEGF.

In accordance with yet another aspect of the present invention, the cells affected to express VEGF comprise cells that have been cultured ex vivo and introduced into the ischemia damaged tissue. The cultured cells can comprise autologous cells that have been harvested from the subject to be treated prior to culturing. In another aspect, the cells affected to express VEGF can comprise native cells of the ischemia damaged tissue.

In accordance with another aspect of the present invention, the ischemia damaged tissue can comprise a first concentration of SDF-1. The concentration of SDF-1 in the ischemia damaged tissue can be increased from the first concentration to a second concentration while the concentration of VEGF in the ischemia damaged tissue is increased. The concentration of SDF-1 in the ischemia damaged tissue can be increased by affecting cells in the ischemia damaged tissue to express SDF-1.

In accordance with another aspect of the present invention, the cells can be affected to express SDF-1 by introducing an expression vector into the cells. The expression vector can include a nucleic acid encoding for SDF-1. The cells affected to express SDF-1 can comprise cells that have been cultured ex vivo and introduced into the ischemia damaged tissue. The cells affected to express SDF-1 can be further comprise autologous cells that have been harvested from the subject to be treated prior to culturing. Alternatively, the cells affected to express SDF-1 can comprise native cells of the ischemia damaged tissue.

In accordance with yet another aspect of the present invention, the cells affected to express VEGF in the ischemia damaged tissue can be further affected to express SDF-1 in the ischemia damaged tissue. The cells can be affected to express SDF-1 by introducing an expression vector into the cells. The expression vector can include a nucleic acid encoding for VEGF.

Another aspect of the present invention relates to a method of stimulating stem cell differentiation in infarcted myocardium. In the method, cells can be introduced into the infarcted myocardium. The cells can be affected to express VEGF in the infarcted myocardium. An agent can be administered that mobilizes the stem cells from bone marrow to peripheral blood of the infarcted myocardium. The stem cells can be mobilized from the bone marrow to the peripheral blood while the VEGF is expressed in the infarcted myocardium.

In another aspect of the present invention, the agent that causes the stem cells to mobilize from the bone marrow to the peripheral blood of the infarcted myocardium can be selected from the group consisting of cytokines, chemokines, and chematherapeutic agents. Preferably, the agent can comprise granulocyte colony stimulating factor (G-CSF).

In accordance with another aspect of present invention, the cells can be affected to express VEGF in the infarcted myocardium using gene therapy. The gene therapy can include affecting the cells with an expression vector to express VEGF. The expression vector can include a nucleotide sequence encoding VEGF.

In accordance with yet another aspect of the present invention, the cells that express VEGF in the infarcted myocardium can comprise cells that have been cultured ex vivo prior to introduction into the infarcted myocardium. In a further aspect, the cells affected to express VEGF can comprise autologous cells that have been harvested from the subject to be treated prior to culturing.

In another aspect of the present invention, the infarcted myocardium can include a first concentration of VEGF. The cells affected to express VEGF can increase the concentration of VEGF in the ischemia damaged tissue from the first concentration to a second concentration.

In accordance with yet another aspect of the present invention, the cells affected to express VEGF in the infarcted myocardium can be further affected to express SDF-1 in the infarcted myocardium. The cells can be affected to express SDF-1 by introducing an expression vector into the cells. The expression vector can include a nucleic acid encoding for SDF-1.

A further aspect of the present invention relates to method of stimulating stem cell differentiation in infarcted myocardium to promote tissue regeneration of the infarcted myocardium. In the method, skeletal myoblasts can be introduced into the infarcted myocardium. The skeletal myoblasts can be transfected with an expression vector to express VEGF in the infarcted myocardium. A colony stimulating factor can be administered that mobilizes said stem cells from bone marrow to peripheral blood of the infarcted myocardium. The stem cells can be mobilized from the bone marrow to the peripheral blood while the VEGF is expressed in the infarcted myocardium. The stem cells that are mobilized from the bone marrow can be differentiated into cardiomyocytes.

### BRIEF DESCRIPTION OF THE DRAWINGS

Further features of the present invention will become apparent to those skilled in the art to which the present invention relates from reading the following description of the invention with reference to the accompanying drawings in which:

FIGS. 1(a and b) are graphs showing, respectively, the number BrdU-positive cells within infarct zone (a) and shortening fraction (b) 4 weeks following administration of saline or G-CSF (12 weeks following LAD ligation).

FIGS. 2(a and b) are graphs showing the effect of skeletal myoblast (SKMB) transplantation on BrdU+ cell counts within the infarct zone 4 weeks following cell transplantation (12 weeks following LAD ligation).

FIGS. 3(a and b) are photographs showing (a) bone marrow stained for BrdU and (b) untreated myocardium after 5 days of BrdU administration.

FIG. 3c is a graph showing the increased BrdU+ cells within the infarct zone assessed with the therapy in accordance with the present invention.

FIG. 4 is a photograph showing RT-PCR revealing stromal derived factor-1 (SDF-1) expression as a function of time following myocardial infarction.

FIGS. 5(a and b) are graphs showing the number of (a) BrdU+cells and (b) CD117+ cells within the infarct zone 4 weeks following transplantation of cardiac fibroblasts stably transfected with or without SDF-1 expression vector with or without G-CSF administration for 5 days following cardiac fibroblast transplantation.

FIG. 5c is a photograph from a SDF-1/G-CSF treated animal stained CD117+.

FIGS. 6(a and b) are photographs showing the immunohistochemistry of the infarct zone revealing both BrdU+ cells and cardiac myosin-expressing cells 2 weeks following LAD ligation with cell transplantation of (a) SKMB or (b) VEGF-expressing SKMB followed by stem cell mobilization using G-CSF.

FIG. 6c is a graph showing improvement in left ventricle function relative cell therapy without VEGF over-expression.

FIGS. 7(a and b) are graphs comparing vascular density (a) and left ventricular function (b) before and after SKMB transplantation.

FIG. 8 is a graph comparing vascular density after direct adenoviral injection and transplantation of cells expressing VEGF-165.

FIGS. 9(a-f) are photographs showing representative sections of the infarct zone 4 weeks following injection into the peri-infarct zone of 1 million SKMB transfected with AdLUC (a, d), 1×10⁷ pfu AdVEGF-165 (b, e), and 1 million SKMB transfected with AdVEGF-165 (c, f) in five equally divided injections.

FIGS. 10(a and b) are photographs showing inflammatory infiltrate in the peri-infarct zone 4 weeks following injection of (a) 1×10⁷ pfu AdVEGF-165 and (b) 1 million SKMB transfected with 1×10⁷ pfu AdVEGF-165 each in five equally divided injections.

FIGS. 11(a and b) are graphs showing the effect of transplantation of VEGF-165 expressing SKMB on left ventricle (LV) function, presented as (a) shortening fraction (%) and (b) relative to saline control.

### DESCRIPTION OF THE EMBODIMENTS

Unless otherwise defined, all technical terms used herein have the same meaning as commonly understood by one of ordinary skill in the art to which this invention belongs. Commonly understood definitions of molecular biology terms can be found in, for example, Rieger et al., Glossary of Genetics: Classical and Molecular, 5th edition, Springer-Verlag: New York, 1991; and Lewin, Genes V, Oxford University Press: New York, 1994.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

The present invention relates to a method of stimulating stem cell differentiation in ischemia damaged tissue to regenerate the ischemia damaged tissue. The method of the present invention can be used to treat ischemia damaged tissue at a time remote (i.e., weeks) from the ischemia.

The method includes mobilizing migration of pluripotent stem cells to ischemia damaged tissue within a mammalian subject. Pluripotent stem cells described in the invention can be any cells that can be stimulated by the method of the present invention to differentiate into another cell type. One example of pluripotent stem cells includes hematopoietic stem cells that can differentiate into cardiomyocyte cells.

Mammalian subjects can include any mammal, such as human beings, rats, mice, cats, dogs, goats, sheep, horses, monkeys, apes, rabbits, cattle, etc. The mammalian subject can be in any stage of development including adults, young animals, and neonates. Mammalian subjects can also include those in a fetal stage of development.

The ischemia damaged tissue can include any tissue that is damaged by deficiency of blood supply to the tissue. The deficiency of blood supply can be caused by occlusion or stenosis of the blood supplying artery or by occlusion or stenosis of the vein that drains the tissue.

In one aspect of the present invention, the ischemia damaged tissue can include infarcted myocardium. Infarcted myocardium as used in the present invention refers to infarcted myocardial tissue, peripheral tissue of the infarcted myocardial tissue (e.g., skeletal muscle tissue in the case of peripheral vascular tissue), and both the infarcted myocardial tissue and peripheral tissue of the infarcted myocardial tissue.

At a time remote from the ischemia, a first number of pluripotent stem cells traffic the ischemia damaged tissue. This first number of stem cells can be increased so that a greater number of stem cells traffic the ischemia damaged. By increasing the number of stem cells that traffic the ischemia damaged tissue, the ischemia damaged tissue can be regenerated because there will be a greater number of pluripotent stem cells in the ischemia damaged tissue that can differentiate into cells, which can repopulate (i.e., engraft) and partially or wholly restore the normal function of the ischemia damaged tissue.

In another aspect of the present invention, the method includes a step of increasing the concentration of vascular endothelial growth factor (VEGF) in the ischemia damaged tissue from a first concentration to a second concentration substantially greater than the first concentration. The first concentration of VEGF in the ischemia damaged tissue is the concentration of VEGF typically found in the ischemia damaged tissue at a time remote (i.e., weeks) from the ischemia. The concentration of VEGF can be increased in the ischemia damaged tissue by up-regulating the expression of VEGF in the ischemia damaged tissue from the amount of VEGF typically expressed in the ischemia damaged tissue at a time remote from the ischemia.

Increasing the concentration of vascular endothelial growth factor in the ischemia damaged tissue increases vascular density within the ischemia damaged tissue compared to saline controls. The VEGF expressed in the ischemia damaged tissue was also found to stimulate stem cell differentiation and regeneration of the ischemia damaged tissue. For example, VEGF expressed in infarcted myocardium was found to differentiate mobilized stem cells into cardiomyocytes.

The method of the present invention further includes a step of mobilizing stem cells to the ischemia damaged tissue so that the concentration (i.e., number) of pluripotent stems cells in peripheral blood of the ischemia damaged tissue is increased from a first concentration to a second concentration substantially greater than the first concentration. The first concentration of stem cells can be the concentration of stem cells typically found in the peripheral blood at a time remote from the ischemia. The concentration of stem cells in the peripheral blood can be increased either before or after the concentration of VEGF is up-regulated in the ischemia damaged tissue as long as the concentration of stem cells in the peripheral blood is increased while the concentration of VEGF in the ischemia damaged tissue is increased.

In accordance with another aspect of the present invention, the method can include inducing the pluripotent stem cells to home to the ischemia damaged tissue. The pluripotent stem cells can be homed to the ischemia damaged tissue by increasing the concentration of SDF-1 protein within the ischemia damaged tissue from a first concentration to a second concentration substantially greater than the first concentration. The first concentration of SDF-1 protein can be the concentration of SDF-1 protein typically found in an ischemia damaged tissue (e.g., infarcted myocardium) at a time remote (i.e., weeks) from the ischemia (e.g., myocardial infarction). The second concentration of SDF-1 protein can be substantially greater than the first concentration of SDF-1 protein.

The concentration of SDF-1 protein can be increased by up-regulating the expression of SDF-1 protein within the ischemia damaged tissue from the amount of SDF-1 protein typically expressed in the ischemia damaged tissue at a time remote from the myocardial infarction. The concentration of SDF-1 in the ischemia damaged tissue can be increased while the concentration of VEGF-1 in the ischemia damaged tissue is increased and the concentration of stem cells in the peripheral blood is increased.

VEGF

In accordance with one aspect of the present invention, the VEGF that is expressed in the ischemia damaged tissue is one of the family of vascular endothelial growth factors that can induce the growth of new collateral blood vessels. VEGFs are specific angiogenic growth factors that have vaso-permeability activity and target endothelial cells almost exclusively.

The VEGF that is expressed in the ischemia damaged tissue can be an expression product of a VEGF gene. Preferred VEGFs that can be used in accordance with the present invention include VEGF-1 (also known as VEGF-A) and other structurally homologous VEGF's, such as VEGF-2 (VEGF-C), VEGF-3 (VEGF-B), VEGF-D, VEGF-E, and placental growth factor. Known isoforms of VEGF-1 can include, for example, 121, 138, 162, 165, 182, 189, and 206 amino acids. These isoforms are identified, respectively, as VEGF-121, VEGF-165, VEGF-162, VEGF-182, VEGF-189, and VEGF-206. The mitogenic and heparin binding activity of these isoforms differ. A preferred isoform of VEGF-1 used in accordance with the present invention is VEGF-165. Other isoforms of VEGF-1 and other homologs of VEGF not listed can also be used in accordance with the present invention.

The VEGF of the present invention can have an amino acid sequence identical to one of the foregoing VEGFs. The VEGF of the present invention can also be a variant of one of the foregoing VEGFs, such as a fragment, analog and derivative of VEGF. Such variants include, for example, a polypeptide encoded by a naturally occurring allelic variant of native VEGF gene (i.e., a naturally occurring nucleic acid that encodes a naturally occurring mammalian VEGF), a polypeptide encoded by an alternative splice form of a native VEGF gene, a polypeptide encoded by a homolog of a native VEGF gene, and a polypeptide encoded by a non-naturally occurring variant of a VEGF gene.

VEGF variants have a peptide sequence that differs from a native VEGF in one or more amino acids. The peptide sequence of such variants can feature a deletion, addition, or substitution of one or more amino acids of a native VEGF. Amino acid insertions are preferably of about 1 to 4 contiguous amino acids, and deletions are preferably of about 1 to 10 contiguous amino acids. Variant VEGF in accordance with the present invention substantially maintain a native VEGF functional activity. Preferred VEGF protein variants can be made by expressing nucleic acid molecules within the invention that feature silent or conservative changes.

VEGF fragments corresponding to one or more particular motifs and/or domains or to arbitrary sizes, are within the scope of the present invention. Isolated peptidyl portions of VEGF can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry.

Variants of VEGF can also include recombinant forms of VEGF. Recombinant polypeptides preferred by the present invention, in addition to a VEGF, are encoded by a nucleic acid that has at least 85% sequence identity with the nucleic acid sequence of a gene encoding a mammalian VEGF.

VEGF variants can include agonistic forms of the protein that constitutively express the functional activities of a native VEGF. Other VEGF variants can include those that are resistant to proteolytic cleavage, as for example, due to mutations which alter protease target sequences. Whether a change in the amino acid sequence of a peptide results in a variant having one or more functional activities of a VEGF can be readily determined by testing the variant for VEGF functional activity.

VEGF Nucleic Acids

Another aspect of the present invention relates to nucleic acid molecules that encode VEGF and non-native nucleic acids that encode a mammalian VEGF. Such nucleic acid molecules may be in the form of RNA or in the form of DNA (e.g., cDNA, genomic DNA, and synthetic DNA). The DNA may be double-stranded or single-stranded, and if single-stranded may be the coding (sense) strand or non-coding (anti-sense) strand.

Other nucleic acid molecules within the scope of the invention are variants of a native VEGF gene, such as those that encode fragments, analogs and derivatives of a native VEGF. Such variants may be, for example, a naturally occurring allelic variant of a VEGF gene, a homolog of a native VEGF gene, or a non-naturally occurring variant of a native VEGF gene. These variants have a nucleotide sequence that differs from a native VEGF gene in one or more bases. For example, the nucleotide sequence of such variants can feature a deletion, addition, or substitution of one or more nucleotides of a native VEGF gene. Nucleic acid insertions are preferably of about 1 to 10 contiguous nucleotides, and deletions are preferably of about 1 to 30 contiguous nucleotides.

In other applications, variant VEGF displaying substantial changes in structure can be generated by making nucleotide substitutions that cause less than conservative changes in the encoded polypeptide. Examples of such nucleotide substitutions are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamine or aspartine; or (d) a residue having a bulky side chain, e.g., phenylalanine, for (or by) one not having a side chain, e.g., glycine.

Naturally occurring allelic variants of VEGF gene within the invention are nucleic acids isolated from mammalian tissue that have at least 75% sequence identity with a native VEGF gene, and encode polypeptides having structural similarity to a native VEGF. Homologs of a native VEGF within the invention are nucleic acids isolated from other species that have at least 75% sequence identity with the native gene, and encode polypeptides having structural similarity to a native VEGF. Public and/or proprietary nucleic acid databases can be searched to identify other nucleic acid molecules having a high percent sequence identity to a native VEGF gene.

Non-naturally occurring VEGF variants are nucleic acids that do not occur in nature (e.g., are made by the hand of man), have at least 75% sequence identity with a native VEGF gene, and encode polypeptides having structural similarity to a native VEGF. Examples of non-naturally occurring VEGF gene variants are those that encode a fragment of a native VEGF, those that hybridize to a native VEGF gene or a complement of to a native VEGF gene under stringent conditions, those that share at least 65% sequence identity with a native VEGF gene or a complement of a native VEGF gene, and those that encode a VEGF.

Nucleic acids encoding fragments of VEGF within the invention are those that encode amino acid residues of a native VEGF. Shorter oligonucleotides that encode or hybridize with nucleic acids that encode fragments of a native VEGF can be used as probes, primers, or antisense molecules. Longer polynucleotides that encode or hybridize with nucleic acids that encode fragments of a native VEGF can also be used in various aspects of the invention. Nucleic acids encoding fragments of a native VEGF can be made by enzymatic digestion (e.g., using a restriction enzyme) or chemical degradation of the full length native VEGF gene or variants thereof.

Nucleic acids that hybridize under stringent conditions to one of the foregoing nucleic acids can also be used in the invention. For example, such nucleic acids can be those that hybridize to one of the foregoing nucleic acids under low stringency conditions, moderate stringency conditions, or high stringency conditions.

Nucleic acid molecules encoding a VEGF fusion protein may also be used in the invention. Such nucleic acids can be made by preparing a construct (e.g., an expression vector) that expresses a VEGF fusion protein when introduced into a suitable target cell. For example, such a construct can be made by ligating a first polynucleotide encoding VEGF fused in frame with a second polynucleotide encoding another protein such that expression of the construct in a suitable expression system yields a fusion protein.

The oligonucleotides of the invention can be DNA or RNA or chimeric mixtures or derivatives or modified versions thereof, single-stranded or double-stranded. Such oligonucleotides can be modified at the base moiety, sugar moiety, or phosphate backbone, for example, to improve stability of the molecule, hybridization, etc. Oligonucleotides within the invention may additionally include other appended groups such as peptides (e.g., for targeting cell receptors in vivo), or agents facilitating transport across the cell membrane. To this end, the oligonucleotides may be conjugated to another molecule, e.g., a peptide, hybridization triggered cross-linking agent, transport agent, hybridization-triggered cleavage agent, etc.

VEGF Expression

In accordance with another aspect of present invention, the expression VEGF in ischemia damaged tissue can be increased by introducing an agent into target cells that increases expression of VEGF. The target cells can include those cells within the ischemia damaged tissue or ex vivo cells which are transplanted into the ischemia damaged tissue following introduction of the agent.

The ex vivo cells can be any cells that are biocompatible with the tissue in which the cells are to be transplanted. These cells are preferably harvested from the subject to be treated (i.e., autologous cells) and cultured prior to transplantation. Autologous cells are preferred in order to increase the biocompatibility of the cells upon transplantation and minimize the likelihood of rejection.

Where the ischemia damaged tissue comprises infarcted myocardium, the cells transplanted into the infarcted myocardium can be, for example, autologous, cultured skeletal myoblasts, fibroblasts, smooth muscle cells, and bone marrow derived cells. Preferred cells for transplantation into the infarcted myocardium are skeletal myoblasts. Myoblasts maintain the regenerative potential of skeletal muscle, during periods of stress, proliferate and differentiate into myotubes, eventually forming muscle fibers capable of contracting. Myoblasts implanted into myocardium undergo myotube formation, withdraw from cell cycle, and remain viable. Functional studies have shown an improvement in regional contractility and compliance after myoblast implantation into the myocardium. Skeletal myoblasts can be readily harvested under the basal membrane of muscular fibers, cultured to scale up the cell line, and then transplanted into infarcted myocardium. For example, in a murine subject, skeletal myoblasts can be harvested from the hind limbs of the subject, cultured, and then transplanted into the infarcted myocardium of the subject.

The agent that is introduced into the target cell to increase the expression of VEGF can comprise natural or synthetic VEGF nucleic acids that can be incorporated into recombinant nucleic acid constructs, typically DNA constructs, capable of introduction into and replication in the cell. Such a construct preferably includes a replication system and sequences that are capable of transcription and translation of a polypeptide-encoding sequence in a given target cell.

Other agents can also be introduced into the target cells to increase VEGF levels in the target cells. For example, agents that increase the transcription of a gene encoding VEGF; increase the translation of an mRNA encoding VEGF, and/or those that decrease the degradation of an mRNA encoding VEGF could be used to increase VEGF levels. Increasing the rate of transcription from a gene within a cell can be accomplished by introducing an exogenous promoter upstream of the gene encoding VEGF. Enhancer elements which facilitate expression of a heterologous gene may also be employed.

A preferred method of introducing the agent into a target cell involves using gene therapy. Gene therapy is refers to gene transfer to express a therapeutic product from a cell in vivo or in vitro. Gene therapy in accordance with the present invention can be used to express VEGF from a target cell in vivo or in vitro.

One method of gene therapy uses a vector including a nucleotide encoding VEGF. A “vector” (sometimes referred to as gene delivery or gene transfer “vehicle”) refers to a macromolecule or complex of molecules comprising a polynucleotide to be delivered to a target cell, either in vitro or in vivo. The polynucleotide to be delivered may comprise a coding sequence of interest in gene therapy. Vectors include, for example, viral vectors (such as adenoviruses (‘Ad’), adeno-associated viruses (AAV), and retroviruses), liposomes and other lipid-containing complexes, and other macromolecular complexes capable of mediating delivery of a polynucleotide to a target cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bi-functional. Positive selectable markers allow selection for cells carrying the marker, whereas negative selectable markers allow cells carrying the marker to be selectively eliminated. A variety of such marker genes have been described, including bi-functional (i.e. positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

Vectors for use in the present invention include viral vectors, lipid based vectors and other vectors that are capable of delivering a nucleotide according to the present invention to the target cells. The vector can be a targeted vector, especially a targeted vector that preferentially binds to the target cells (e.g., cardiomyocytes). Preferred viral vectors for use in the invention are those that exhibit low toxicity to a target cell and induce production of therapeutically useful quantities of VEGF in a tissue or cell specific manner.

Presently preferred viral vectors are derived from adenovirus (Ad) or adeno-associated virus (AAV). Both human and non-human viral vectors can be used, but preferably the recombinant viral vector is replication-defective in humans. Where the vector is an adenovirus, it preferably comprises a polynucleotide having a promoter operably linked to a gene encoding the VEGF and is replication-defective in humans.

Adenovirus vectors are preferred for use in the invention because they (1) are capable of highly efficient gene expression in target cells and (2) can accommodate a relatively large amount of heterologous (non-viral) DNA. A preferred form of recombinant adenovirus is a “gutless, “high-capacity”, or “helper-dependent” adenovirus vector. Such a vector features, for example, (1) the deletion of all or most viral-coding sequences (those sequences encoding viral proteins), (2) the viral inverted terminal repeats (ITRs) which are sequences required for viral DNA replication, (3) up to 28-32 kb of “exogenous” or “heterologous” sequences (e.g., sequences encoding a SDF-1 protein), and (4) the viral DNA packaging sequence which is required for packaging of the viral genomes into infectious capsids. For specifically myocardial cells, preferred variants of such recombinant adenoviral vectors contain tissue-specific (e.g., cardiomyocyte) enhancers and promoters operably linked to a VEGF gene.

AAV-based vectors are advantageous because they exhibit high transduction efficiency of target cells and can integrate into the target genome in a site-specific manner. Use of recombinant AAV vectors is discussed in detail in Tal, J., J. Biomed. Sci. 7:279-291, 2000 and Monahan and Samulski, Gene Therapy 7:24-30, 2000. A preferred AAV vector comprises a pair of AAV inverted terminal repeats which flank at least one cassette containing a tissue (e.g., myocardium)—or cell (e.g., cardiomyocyte)—specific promoter operably linked to a VEGF nucleic acid. The DNA sequence of the AAV vector, including the ITRs, the promoter and VEGF gene may be integrated into the target genome.

Other viral vectors that can be use in accordance with the present invention include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid. A preferred HSV vector is one that: (1) is engineered from HSV type I, (2) has its IE genes deleted, and (3) contains a tissue-specific (e.g., myocardium) promoter operably linked to a VEGF nucleic acid. HSV amplicon vectors may also be useful in various methods of the invention. Typically, HSV amplicon vectors are approximately 15 kb in length, and possess a viral origin of replication and packaging sequences.

Retroviruses, such as C-type retroviruses and lentiviruses, might also be used in the invention. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and an SDF-1 nucleic acid. In methods of delivery to an infarcted myocardium, it may also encode a ligand to a myocardial specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells.

Lentiviral vectors for use in the invention may be derived from human and non-human (including SIV) lentiviruses. Preferred lentiviral vectors include nucleic acid sequences required for vector propagation as well as a tissue-specific promoter (e.g., myocardium) operably linked to a VEGF gene. These former vectors may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000. Alphavirus vectors typically are constructed in a format known as a replicon. A replicon may contain (1) alphavirus genetic elements required for RNA replication, and (2) a heterologous nucleic acid such as one encoding a VEGF nucleic acid. Within an alphavirus replicon, the heterologous nucleic acid may be operably linked to a tissue-specific (e.g., myocardium) promoter or enhancer.

Recombinant, replication-defective alphavirus vectors are advantageous because they are capable of high-level heterologous (therapeutic) gene expression, and can infect a wide target cell range. Alphavirus replicons may be targeted to specific cell types (e.g., cardiomyocytes) by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell. A preferred alphavirus vector or replicon is non-cytopathic.

In many of the viral vectors compatible with methods of the invention, more than one promoter can be included in the vector to allow more than one heterologous gene to be expressed by the vector. Further, the vector can comprise a sequence which encodes a signal peptide or other moiety which facilitates the expression of the VEGF gene product from the target cell.

To combine advantageous properties of two viral vector systems, hybrid viral vectors may be used to deliver a VEGF nucleic acid to a target tissue (e.g., myocardium). Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the target cell genome and affect stable VEGF gene expression. Other nucleotide sequence elements, which facilitate expression of the VEGF gene and cloning of the vector are further contemplated. For example, the presence of enhancers upstream of the promoter or terminators downstream of the coding region, for example, can facilitate expression.

The present invention also contemplates the use of tissue-specific promoters for cell targeting. For example, where the ischemia damaged tissue comprises infarcted myocardium, tissue-specific transcriptional control sequences of left ventricular myosin light chain-2 (MLC₂ᵥ) or myosin heavy chain (MHC) can be fused to a transgene, such as the VEGF-165 gene within the adenoviral construct. By fusing such tissue-specific transcriptional control sequences to the transgene, transgene expression can be limited to ventricular cardiomyocytes. By using the MLC₂ᵥ, or MHC promoters and delivering the transgene in vivo, it is believed that the cardiomyocyte alone (that is without concomitant expression in endothelial cells, smooth muscle cells, and fibroblasts within the heart) will provide adequate expression of an angiogenic protein, such as VEGF-165 to promote angiogenesis.

Limiting expression to cardiomyocytes also has advantages regarding the utility of gene transfer for treatment of congestive heart failure. By limiting expression to the heart, one avoids the potentially harmful effect of angiogenesis in non-cardiac tissues. In addition, of the cells in the heart, the myocyte would likely provide the longest transgene expression since the cells do not undergo rapid turnover; expression would not therefore be decreased by cell division and death as would occur with endothelial cells. Endothelial-specific promoters are already available for this purpose.

In addition to viral vector-based methods, non-viral methods may also be used to introduce a VEGF gene into a target cell. A preferred non-viral gene delivery method according to the invention employs plasmid DNA to introduce a VEGF nucleic acid into a cell. Plasmid-based gene delivery methods are generally known in the art.

Synthetic gene transfer molecules can be designed to form multimolecular aggregates with plasmid DNA (e.g., harboring a VEGF coding sequence operably linked to a myocardium-specific promoter). These aggregates can be designed to bind to a target cell (e.g., cardiomyocyte).

Cationic amphiphiles, including lipopolyamines and cationic lipids, may be used to provide receptor-independent VEGF nucleic acid transfer into target cells (e.g., cardiomyocytes). In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the invention. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

Vectors that encode the expression of VEGF can be delivered to the target cell in the form of an injectable preparation containing pharmaceutically acceptable carrier, such as saline, as necessary. Other pharmaceutical carriers, formulations and dosages can also be used in accordance with the present invention. Where the target cell comprises a cell of the ischemia damaged tissue, the vector can be delivered by direct injection using a tuberculin syringe under fluoroscopic guidance, at an amount sufficient for the VEGF to be expressed to a degree which allows for effective stimulation of stem cell differentiation. By injecting the vector directly into the ischemia damaged tissue, it is possible to target the gene rather effectively, and to minimize loss of the recombinant vectors.

This type of injection enables local transfection of a desired number of cells, especially cardiomyocytes in an infarcted myocardium, thereby maximizing therapeutic efficacy of gene transfer, and minimizing the possibility of an inflammatory response to viral proteins. Where the ischemia damaged tissue comprises infarcted myocardium, a cardiomyocyte-specific promoter may be used, for example, to securely enable expression limited to cardiomyocytes. Thus, delivery of the transgenes in this matter may result in targeted gene expression in, for example, the cells of the left ventricle (LV). Other techniques well known in the art can also be used for transplanting the vector to the target cells of the infarcted myocardium.

Where the target cell is a cultured cell that is later transplanted into the ischemia damaged tissue, the vectors can be delivered by direct injection into the culture medium. A VEGF encoding nucleic acid transfected into cells may be operably linked to any suitable regulatory sequence, including a tissue specific promoter and enhancer. The transfected target cells can then be transplanted into the ischemia damaged tissue by well known transplantation techniques, such as by direct intracoronary injection using a tuberculin syringe.

Where the ischemia damaged tissue comprises infarcted myocardium, the target cell is preferably an autologous cell that is harvested from the subject to be treated and cultured ex vivo. By first transfecting the target cells ex vivo and than transplanting the transfected target cells to the infarcted myocardium, it was found that the possibility of inflammatory response in the infarcted myocardium was minimized and that the left ventricular function was improved compared to direct injection of the vector into the infarcted myocardium. It is believed that this improvement results from the absence of inflammatory response typically associated with adenoviral injection.

VEGF of the present invention may be expressed for any suitable length of time within the target cell, including transient expression and stable, long-term expression. In a preferred embodiment, the VEGF nucleic acid will be expressed in therapeutic amounts for a suitable and defined length of time.

A therapeutic amount is an amount, which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. Specific dosages of proteins, nucleic acids, or small molecules) can be determined readily determined by one skilled in the art using the experimental methods described below.

Whether the VEGF expression is transient or long-term, it is desirable that the concentration of VEGF expressed in the ischemia damaged tissue be limited to prevent the formation of hemangiomas or endothelial cell-derived intravascular tumors.

Stem Cell Mobilization

In accordance with another aspect of the present invention, the concentration of the stem cells in the peripheral blood of the ischemia damaged tissue of the subject can be increased by administering an agent to induce mobilization of stem cells to the peripheral blood. The stem cells can be mobilized to the peripheral blood of the subject to increase stem cell concentration in peripheral blood using a number of agents. For example, to increase the number of stem cells in the peripheral blood of a mammalian subject, an agent that causes a pluripotent stem cell to mobilize from the bone marrow can be administered to the subject. A number of such agents are known and can include, for example, cytokines, such as granulocyte-colony stimulating factor (G-CSF), granulocyte-macrophage colony stimulating factor (GM-CSF), interleukin (IL)-7, IL-3, IL-12, stem cell factor (SCF), and flt-3 ligand; chemokines, such as IL-8, IL-10, Mip-1α, and Groβ, and the chematherapeutic agents of cylcophosamide (Cy) and paclitaxel. These agents differ in their time frame to achieve stem cell mobilization, the type of stem cell mobilized, and efficiency.

The mobilizing agent can be administered by direct injection of the mobilizing agent into the subject. Preferably, the mobilizing agent is administered after VEGF expression is up-regulated in the ischemia damaged tissue. The mobilizing agent, however, can be administered before VEGF expression is up-regulated.

A preferred mobilizing agent is a colony stimulating factor, such as G-CSF. A typical dosage of G-CSF in a murine subject is about 125 μg/Kg per day for about 5 to about 10 days. The G-CSF agent can be administered after the VEGF expression is up-regulated.

Alternatively, as is well known in the art, the concentration of stem cells in the peripheral blood can be increased by the injection of specialized stem cells (i.e., MAPC's) into the peripheral blood.

SDF-1 Protein

In accordance with another aspect of the present invention, the SDF-1 protein (or SDF-1 polypeptide) that is expressed in the ischemia damaged tissue can be an expression product of an SDF-1 gene. The amino acid sequence of a number or different mammalian SDF-1 proteins are known including, for example, human, rat, mouse, and cat. The SDF-1 protein can have an amino acid sequence identical to one of the foregoing native mammalian SDF-1 proteins.

The SDF-1 protein of the present invention can also be a variant of mammalian SDF-1 protein, such as a fragment, analog and derivative of mammalian SDF-1 protein. Such variants include, for example, a polypeptide encoded by a naturally occurring allelic variant of native SDF-1 gene (i.e., a naturally occurring nucleic acid that encodes a naturally occurring mammalian SDF-1 protein), a polypeptide encoded by an alternative splice form of a native SDF-1 gene, a polypeptide encoded by a homolog of a native SDF-1 gene, and a polypeptide encoded by a non-naturally occurring variant of a native SDF-1 gene.

SDF-1 protein variants have a peptide sequence that differs from a native SDF-1 protein in one or more amino acids. The peptide sequence of such variants can feature a deletion, addition, or substitution of one or more amino acids of a SDF-1 protein. Amino acid insertions are preferably of about 1 to 4 contiguous amino acids, and deletions are preferably of about 1 to 10 contiguous amino acids. Variant SDF-1 proteins substantially maintain a native SDF-1 protein functional activity. Preferred SDF-1 protein variants can be made by expressing nucleic acid molecules within the invention that feature silent or conservative changes.

SDF-1 protein fragments corresponding to one or more particular motifs and/or domains or to arbitrary sizes, are within the scope of the present invention. Isolated peptidyl portions of SDF-1 proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a SDF-1 protein of the present invention may be arbitrarily divided into fragments of desired length with no overlap of the fragments, or preferably divided into overlapping fragments of a desired length. The fragments can be produced recombinantly and tested to identify those peptidyl fragments which can function as agonists of native SDF-1 protein.

Variants of SDF-1 protein can also include recombinant forms of the SDF-1 proteins. Recombinant polypeptides preferred by the present invention, in addition to a SDF-1 protein, are encoded by a nucleic acid that can have at least 85% sequence identity with the nucleic acid sequence of a gene encoding a mammalian SDF-1 protein.

SDF-1 protein variants can include agonistic forms of the protein that constitutively express the functional activities of a native SDF-1 protein. Other SDF-1 protein variants can include those that are resistant to proteolytic cleavage, as for example, due to mutations, which alter protease target sequences. Whether a change in the amino acid sequence of a peptide results in a variant having one or more functional activities of a native SDF-1 protein can be readily determined by testing the variant for a native SDF-1 protein functional activity.

SDF-1 Nucleic Acids

Another aspect of the present invention relates to nucleic acid molecules that encode an SDF-1 protein and non-native nucleic acids that encode an SDF-1 protein. Such nucleic acid molecules may be in the form of RNA or in the form of DNA (e.g., cDNA, genomic DNA, and synthetic DNA). The DNA may be double-stranded or single-stranded, and if single-stranded may be the coding (sense) strand or non-coding (anti-sense) strand. The coding sequence which encodes an SDF-1 protein may be identical to a nucleotide sequence shown GenBank Accession No. AF189724, GenBank Accession No. AF209976, GenBank Accession No. L120029, and GenBank Accession No. NM022177. It may also be a different coding sequence which, as a result of the redundancy or degeneracy of the genetic code, encodes the same polypeptide as such polynucleotides.

Other nucleic acid molecules that encode SDF-1 within the invention are variants of a native SDF-1 such as those that encode fragments, analogs and derivatives of a native SDF-1 protein. Such variants may be, for example, a naturally occurring allelic variant of a native SDF-1 gene, a homolog of a native SDF-1 gene, or a non-naturally occurring variant of a native SDF-1 gene. These variants have a nucleotide sequence that differs from a native SDF-1 gene in one or more bases. For example, the nucleotide sequence of such variants can feature a deletion, addition, or substitution of one or more nucleotides of a native SDF-1 gene. Nucleic acid insertions are preferably of about 1 to 10 contiguous nucleotides, and deletions are preferably of about 1 to 10 contiguous nucleotides.

In other applications, variant SDF-1 proteins displaying substantial changes in structure can be generated by making nucleotide substitutions that cause less than conservative changes in the encoded polypeptide. Examples of such nucleotide substitutions are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamine or aspartine; or (d) a residue having a bulky side chain, e.g., phenylalanine, for (or by) one not having a side chain, e.g., glycine.

Naturally occurring allelic variants of a native SDF-1 gene within the invention are nucleic acids isolated from mammalian tissue that have at least 75% sequence identity with a native SDF-1 gene, and encode polypeptides having structural similarity to a native SDF-1 protein. Homologs of a native SDF-1 gene within the invention are nucleic acids isolated from other species that have at least 75% sequence identity with the native gene, and encode polypeptides having structural similarity to a native SDF-1 protein. Public and/or proprietary nucleic acid databases can be searched to identify other nucleic acid molecules having a high percent (e.g., 70% or more) sequence identity to a native SDF-1 gene.

Non-naturally occurring SDF-1 gene variants are nucleic acids that do not occur in nature (e.g., are made by the hand of man), have at least 75% sequence identity with a native SDF-1 gene, and encode polypeptides having structural similarity to a native SDF-1 protein. Examples of non-naturally occurring SDF-1 gene variants are those that encode a fragment of a native SDF-1 protein, those that hybridize to a native SDF-1 gene or a complement of to a native SDF-1 gene under stringent conditions, those that share at least 65% sequence identity with a native SDF-1 gene or a complement of a native SDF-1 gene, and those that encode a SDF-1 fusion protein.

Nucleic acids encoding fragments of a native SDF-1 protein within the invention are those that encode, amino acid residues of a native SDF-1 protein. Shorter oligonucleotides that encode or hybridize with nucleic acids that encode fragments of a native SDF-1 protein can be used as probes, primers, or antisense molecules. Longer polynucleotides that encode or hybridize with nucleic acids that encode fragments of a native SDF-1 protein can also be used in various aspects of the invention. Nucleic acids encoding fragments of a native SDF-1 can be made by enzymatic digestion (e.g., using a restriction enzyme) or chemical degradation of the full length native SDF-1 gene or variants thereof.

Nucleic acids that hybridize under stringent conditions to one of the foregoing nucleic acids can also be used in the invention. For example, such nucleic acids can be those that hybridize to one of the foregoing nucleic acids under low stringency conditions, moderate stringency conditions, or high stringency conditions are within the invention.

Nucleic acid molecules encoding a SDF-1 fusion protein may also be used in the invention. Such nucleic acids can be made by preparing a construct (e.g., an expression vector) that expresses a SDF-1 fusion protein when introduced into a suitable target cell. For example, such a construct can be made by ligating a first polynucleotide encoding a SDF-1 protein fused in frame with a second polynucleotide encoding another protein such that expression of the construct in a suitable expression system yields a fusion protein.

The oligonucleotides of the invention can be DNA or RNA or chimeric mixtures or derivatives or modified versions thereof, single-stranded or double-stranded. Such oligonucleotides can be modified at the base moiety, sugar moiety, or phosphate backbone, for example, to improve stability of the molecule, hybridization, etc. Oligonucleotides within the invention may additionally include other appended groups such as peptides (e.g., for targeting target cell receptors in vivo), or agents facilitating transport across the cell membrane, hybridization-triggered cleavage. To this end, the oligonucleotides may be conjugated to another molecule, e.g., a peptide, hybridization triggered cross-linking agent, transport agent, hybridization-triggered cleavage agent, etc.

SDF-1 Expression

In accordance with another aspect of present invention, the expression of SDF-1 in the ischemia damaged tissue can be upregulated by the introduction cells into the ischemia damaged tissue. The introduction of cell into ischemia damaged tissue up-regulates the expression of SDF-1 protein in the ischemia damaged tissue. For example, skeletal myoblasts transplanted into infarcted myocardium of a murine subject up-regulates the expression of SDF-1 protein in the infarcted myocardium from about 1 hour after transplantation of the skeletal myoblasts into the infarcted myocardium to less than about 7 days after transplantation.

Cell types that can be transplanted into the ischemia damaged tissue include any cells that are biocompatible with the tissue in which the cells are to be transplanted. These cells are preferably harvested from the subject to be treated (i.e., autologous cells) and cultured prior to transplantation. Autologous cells are preferred in order to increase the biocompatibility of the cells upon transplantation and minimize the likelihood of rejection.

Where the ischemia damaged tissue comprises infarcted myocardium the cells transplanted into the infarcted myocardium can be, for example, aulogous, cultured skeletal myoblasts, fibroblasts, smooth muscle cells, and bone marrow derived cells. Preferred cells for transplantation into the infarcted myocardium are skeletal myoblasts.

The cells that are introduced into the ischemia damaged tissue to up-regulate the expression of SDF-1 protein can be the same cells that can be introduced into the ischemia damaged tissue to express VEGF or can be different cells. Where the VEGF concentration is increased by introducing into the ischemia damaged tissue cells expressing VEGF, the cells expressing VEGF are preferably the same cells used to upregulate the expression of SDF-1 protein in the ischemia damaged tissue. For example, a skeletal myoblast transfected to express VEGF when transplanted into murine infarcted myocardium will cause transient expression of SDF-1 protein in the infarcted myocardium from about 1 hour after transplantation of the skeletal myoblasts into the infarcted myocardium to less than about 7 days after transplantation.

In another aspect of the present invention, the expression of SDF-1 protein can be upregulated by introducing an agent into target cells that increases the expression of SDF-1 protein in the target cells. The target cells can include cells within the ischemia damaged tissue or ex vivo cells, such as autologous cells, that have been harvested from the subject and cultured. For example, the ex vivo cells can be cells that are introduced into the ischemia damaged tissue to express VEGF and/or cells that are introduced in the ischemia damaged tissue to provide transient expression of SDF-1.

The agent can comprise natural or synthetic nucleic acids, according to present invention and described above, that are incorporated into recombinant nucleic acid constructs, typically DNA constructs, capable of introduction into and replication in the cell. Such a construct preferably includes a replication system and sequences that are capable of transcription and translation of a polypeptide-encoding sequence in a given target cell.

Other agents can also be introduced into the target cells to increase SDF-1 protein levels in the target tissue. For example, agents that increase the transcription of a gene encoding SDF-1 protein increase the translation of an mRNA encoding SDF-1 protein, and/or those that decrease the degradation of an mRNA encoding SDF-1 protein could be used to increase SDF-1 protein levels. Increasing the rate of transcription from a gene within a cell can be accomplished by introducing an exogenous promoter upstream of the gene encoding SDF-1 protein. Enhancer elements which facilitate expression of a heterologous gene may also be employed.

A preferred method of introducing the agent into a target cell involves using gene therapy. Gene therapy in accordance with the present invention can be used to express SDF-1 protein from a target cell in vivo or ex vivo. A preferred gene therapy method involves using a vector including a nucleotide encoding SDF-1. Examples of vectors that can be used include viral vectors (such as adenoviruses (‘Ad’), adeno-associated viruses (AAV), and retroviruses), liposomes and other lipid-containing complexes, and other macromolecular complexes capable of mediating delivery of a polynucleotide to a target cell.

The vectors can also comprise other components or functionalities that further modulate gene delivery and/or gene expression, or that otherwise provide beneficial properties to the targeted cells. Such other components are described above.

Vectors for use in expressing SDF-1 protein include viral vectors, lipid based vectors and other vectors that are capable of delivering a nucleotide according to the present invention to the target cells. The vector can be a targeted vector, especially a targeted vector that preferentially binds to a specific cell type. Preferred viral vectors for use in the invention are those that exhibit low toxicity to a target cell and induce production of therapeutically useful quantities of SDF-1 protein in a tissue-specific manner.

Presently preferred viral vectors are derived from adenovirus (Ad) or adeno-associated virus (AAV). Both human and non-human viral vectors can be used but preferably the recombinant viral vector is replication-defective in humans. Where the vector is an adenovirus, it preferably comprises a polynucleotide having a promoter operably linked to a gene encoding the SDF-1 protein and is replication-defective in humans. Other vectors including viral and non-viral vectors well known in the art and described above can also be used.

In many of the viral vectors compatible with methods of the invention, more than one promoter can be included in the vector to allow more than one heterologous gene to be expressed by the vector. Further, the vector can comprise a sequence which encodes a signal peptide or other moiety which facilitates the secretion of a SDF-1 gene product from the target cell.

To combine advantageous properties of two viral vector systems, hybrid viral vectors may be used to deliver a SDF-1 nucleic acid to a target tissue (e.g., myocardium). Standard techniques for the construction of hybrid vectors are well-known to those skilled in the art. For example, the presence of enhancers upstream of the promoter or terminators downstream of the coding region, for example, can facilitate expression.

In accordance with another aspect of the present invention, a tissue-specific or drug-regulatable promoter can be fused to a SDF-1 gene. By fusing such tissue specific promoter within the adenoviral construct, transgene expression is limited to specific cell types (e.g., ventricular cardiomyocytes) or in response to specific drugs (e.g., tetracycline). The efficacy of gene expression and degree of specificity provided by tissue specific promoters can be determined, using the recombinant adenoviral system of the present invention.

Where the ischemia damaged tissue is infarcted myocardium, the use of tissue specific promoters directed to cardiomyocytes alone (i.e., without concomitant expression in endothelial cells, smooth muscle cells, and fibroblasts within the heart) when delivering the SDF-1 gene in vivo provides adequate expression of the SDF-1 protein for therapeutic treatment. Limiting expression to the cardiomyocytes also has advantages regarding the utility of gene transfer for the treatment of CHF. In addition, cardiomyocytes would likely provide the longest transgene expression since the cells do not undergo rapid turnover; expression would not therefore be decreased by cell division and death as would occur with endothelial cells.

In addition to viral vector-based methods, non-viral methods may also be used to introduce a SDF-1 gene into a target cell. A preferred non-viral gene delivery method according to the invention employs plasmid DNA to introduce a SDF-1 nucleic acid into a cell. Plasmid-based gene delivery methods are generally known in the art.

Methods that involve both viral and non-viral based components may be used according to the invention. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

Vectors that encode the expression of SDF-1 can be delivered to the target cell in the form of an injectable preparation containing a pharmaceutically acceptable carrier, such as saline, as necessary. Other pharmaceutical carriers, formulations and dosages can also be used in accordance with the present invention.

The vector can be delivered by direct injection using a tuberculin syringe under fluoroscopic guidance, at an amount sufficient for the SDF-1 protein to be expressed to a degree which allows for highly effective therapy. By injecting the vector directly into the ischemia damaged tissue, it is possible to target the gene rather effectively, and to minimize loss of the recombinant vectors. This type of injection also enables local transfection of a desired number of cells thereby maximizing therapeutic efficacy of gene transfer, and minimizing the possibility of an inflammatory response to viral proteins.

Where the ischemia damaged tissue comprises infarcted myocardium, a cardiomyocyte-specific promoter may be used, for example, to securely enable expression limited to the cardiomyocytes. Thus, delivery of the transgenes in this matter may result in targeted gene expression in, for example, the cells of the left ventricle. Other techniques well known in the art can also be used for transplanting the vector to the target cells of the infarcted myocardium.

Where the target cell is a cultured cell that is later transplanted into the ischemia damaged tissue, the vectors can be delivered by direct injection into the culture medium. An SDF-1 encoding nucleic acid transfected into cells may be operably linked to any suitable regulatory sequence, including a myocardium specific promoter and enhancer.

The transfected target cells can then be transplanted to the ischemia damaged tissue by well known transplantation techniques, such as by direct injection using a tuberculin syringe. By first transfecting the target cells in vitro and than transplanting the transfected target cells to the infarcted myocardium, the possibility of inflammatory response in the infarcted myocardium is minimized compared to direct injection of the vector into the ischemia damaged tissue.

SDF-1 nucleic acids of the present invention may be expressed for any suitable length of time within the target cell, including transient expression and stable, long-term expression. In a preferred embodiment, the SDF-1 nucleic acid will be expressed in therapeutic amounts for a suitable and defined length of time. Specific dosages of proteins, nucleic acids, or small molecules can be determined readily determined by one skilled in the art using the experimental methods described below.

The SDF-1 expression may be transient as is the case when a cell that is not transfected with an SDF-1 protein encoding vector is transplanted into the infarcted myocardium. Alternatively, SDF-1 protein expression may be long-term, as is the case where the infarcted myocardium is transfected with an SDF-1 protein encoding vector or where a cell that is transfected with an SDF-1 protein encoding vector is transplanted to the infarcted myocardium.

Long term SDF-1 expression is advantageous because it allows the concentration of stem cells to be increased with a mobilizing agent, such as G-CSF or some other factor, at a time remote from the surgery or procedure that transplanted the cells. In the case where G-CSF is the mobilizing agent, there is a significant increase in neutrophil count, which could cause negative effects in the peri-surgical period, but not days or weeks later. Additionally, long term or chronic up-regulation SDF-1 protein expression would allow multiple attempts at stem cell mobilization. Further, chronic up-regulation in SDF-1 protein expression causes long term homing of stem cells into the ischemia damaged tissue from the peripheral blood without the need of stem cell mobilization.

### EXAMPLES

The present invention is further illustrated by the following series of examples. The examples are provided for illustration and are not to be construed as limiting the scope or content of the invention in any way.

### First Series of Examples

Effect of Stem Cell Mobilization with G-CSF 8 Weeks Following MI

In order to ascertain whether stem cell mobilization by G-CSF leads to myocardial regeneration in rats with established ischemic cardiomyopathy, rats 8 weeks following MI were randomized to receive either recombinant human G-CSF (125 μg/kg/day for 5 days, via i.p. injection) or saline. Blood was obtained 5 days after initiating G-CSF therapy to verify bone marrow stimulation, revealing a tripling of the leukocyte count with G-CSF (37.3+5.3 cells/μl) compared with saline (11.8+4.0 cells/μl) therapy. 5-bromo-2′-deoxyuridine, BrdU, was administered beginning on the final day after G-CSF administration for a total of 14 days in order to label any proliferating cells with in the myocardium.

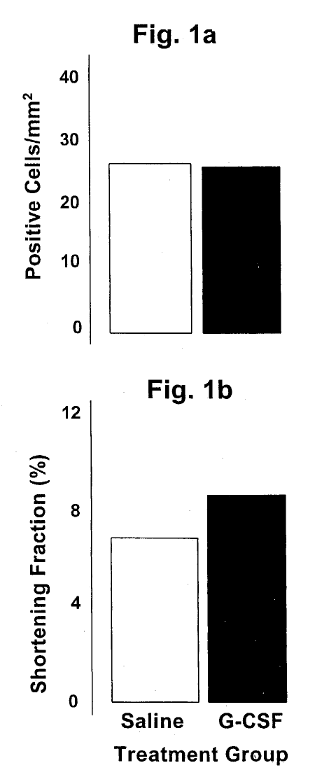

FIGS. 1(a and b) show, respectively, the number BrdU-positive cells within infarct zone (a) and the shortening fraction (b) 4 weeks following administration of saline or G-CSF (12 weeks following LAD ligation). Cell counts are cells per mm². Data represent mean±s.d. n=6-8 per group.

The administration of G-CSF 2 months after LAD ligation did not lead to an increase in BrdU positive cell number within the infarct zone (FIG. 1a) or to meaningful myocardial regeneration as determined by the lack of angiogenesis or cardiac myosin positive cells within the infarct zone (data not shown). Twelve weeks following LAD ligation these animals demonstrated a significant cardiomyopathy with a shortening fraction in control animals of significant less than 10% (normal >60%). Consistent with lack of histological evidence of significant myocardial regeneration in response to G-CSF 8 weeks after LAD ligation, no meaningful recovery of systolic function was seen with G-CSF (FIG. 1b).

Effect of SKMB Transplantation Prior to Stem Cell Mobilization on Ischemic Cardiomyopathy

In order to test the hypothesis that myocardium temporally remote from the time of myocardial infarction can be optimized for myocardial regeneration in response to stem cell mobilization, skeletal myoblast transplantation was performed 8 weeks following LAD ligation. Animals received five injections of 200,000 SKMB/injection within the infarct border zone. Because the initial hypothesis was that the transplanted SKMB would be used as a strategy for expressing gene products responsible for stem cell homing, as a control, the SKMB were transfected with adenovirus encoding luciferase.

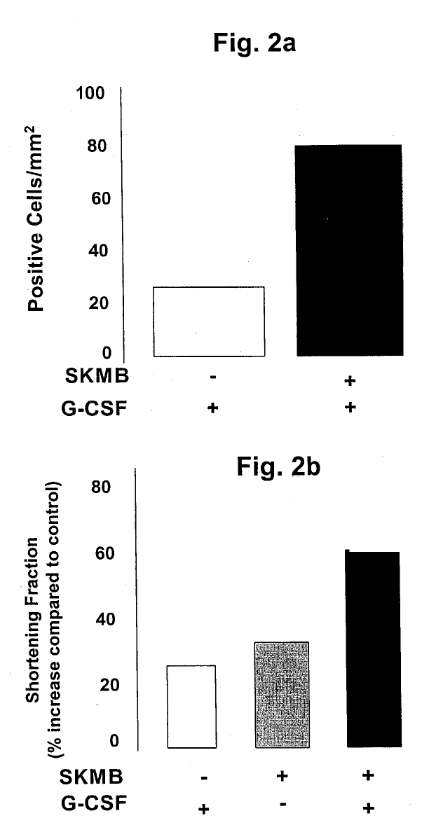

FIGS. 2(a and b) show the effect of skeletal myoblast (SKMB) transplantation on BrdU+ cell counts within the infarct zone four weeks following cell transplantation (12 weeks following LAD ligation). Data represent mean±s.d. n=6-8 per group.

The introduction of SKMB into the infarcted heart in the absence of G-CSF did not significantly increase the incorporation of BrdU positive cells into the infarct zone. However, the combination of SKMB transplantation and G-CSF did result in a significantly increased number of BrdU positive cells within the infarct zone 4 weeks later (FIG. 2a). In addition, compared to animals that received either G-CSF or SKMB transplantation alone, animals that received the combined therapy experienced a significant increase in shortening fraction (FIG. 2b) relative to saline controls.

In order to determine if the BrdU positive cells in the infarct zone originated in the bone marrow, or were endogenous cells from the myocardium that divided, eight weeks following LAD ligation, BrdU was administered, for five days, starting six days prior to transplantation of SKMB and initiation of G-CSF.

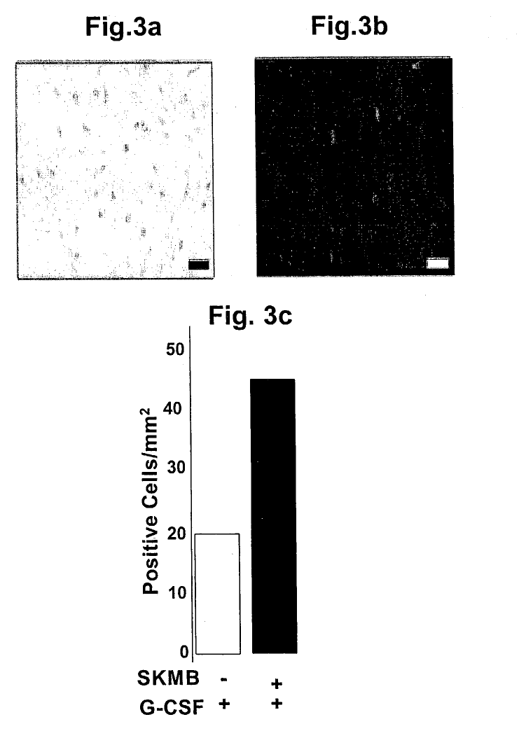

FIG. 3a shows that bone marrow (BM) stained for BrdU revealed almost 100% staining of BM cells cultured from multiple animals. FIG. 3b shows that no significant Brdu+cells were seen in untreated myocardium after five days of BrdU administration. Data represent mean±s.d. of positive cells quantified by two independent observers blinded to the identity of each animal. n=6-8 per group. Scale bar represents 25 μM. This led to labeling of cells in the bone marrow without any significant Brdu labeling of the myocardium (17.5±2.9 positive cells/mm²).

FIG. 3c shows the increased BrdU+cells within the infarct zone assessed with the therapy in accordance with the present invention.

In these experiments, only those animals that received SKMB and G-CSF had a significant increase in the number of BrdU-positive cells within the infarct zone. These data are consistent with the concept that the BrdU-positive cells arose from the bone marrow and homed to the infarct zone following combined therapy. The data also support that SKMB transplantation re-establishes the necessary signals for stem cell homing to the myocardium.

Signaling Molecules Responsible for Stem Cell Homing to Infarcted Tissue

The observation that circulating cells will engraft into infarcted tissue 8 weeks after an MI with “stimulation” of the tissue by SKMB transplantation prompted the evaluation of potential mediators of stem cell homing. Stromal cell-derived factor-1 (SDF-1) is known to mediate hematopoietic trafficking and stem cell homing in the bone marrow; therefore, its role as a potential signaling molecule for stem cell engraftment in MI and in response to SKMB transplantation was assessed.

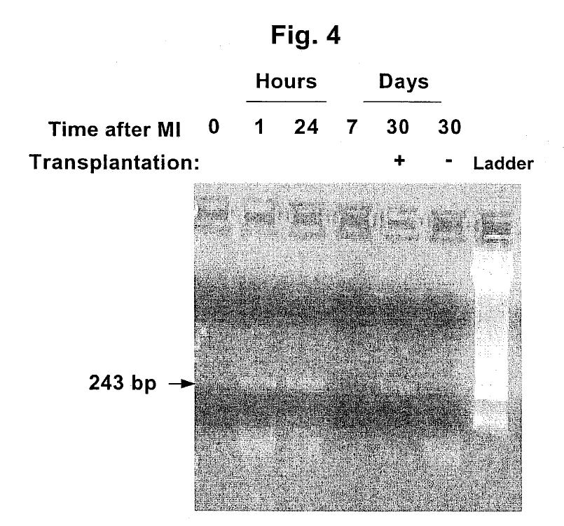

FIG. 4 is a photograph showing RT-PCR revealing stromal derived factor-1 (SDF-1) expression as a function of time following myocardial infarction. SDF-1 expression is absent at baseline, increased at 1 and 24 hours following MI. SDF-1 expression returns to its absent state between 24 hours and 7 days following MI and remains absent 30 days following MI. SDF-1 expression recurs 72 hours following SKMB transplantation performed 30 days (30+) following MI.

Thus, by RT-PCR, SDF-1 expression was observed at 1 and 24 h, but not at 0, 7 or 30 days, after LAD ligation. SDF-1 expression was induced by SKMB, and was observed 72 h after transplantation, but not in sham operated animals (data not shown). PCR for GAPDH in the same samples demonstrated that cDNA was intact in all samples (data not shown). The increase in SDF-1 expression in response to SKMB transplantation was confirmed by real-time PCR (data not shown). Real-time PCR revealed GAPDH levels were similar among groups.

To evaluate whether SDF-1 mediated engraftment of BrdU positive cells into the infarct zone, control cardiac fibroblasts or those stably transfected with an SDF-1 expression vector were transplanted into myocardium 4 weeks following LAD ligation. Ten days later, to allow for down-regulation of endogenous SDF-1 expression, G-CSF was administered for five days, as was BrdU for five days beginning on the final day of G-CSF administration.

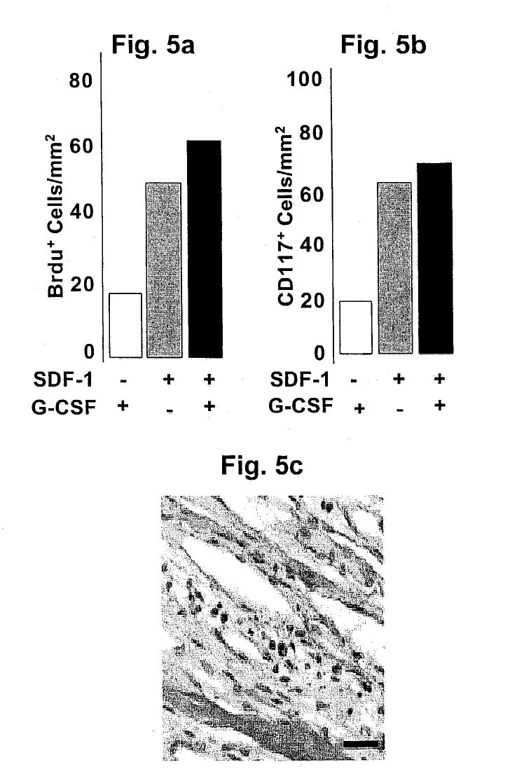

FIG. 5(a and b) show the number of (a) BrdU+cells and (b) CD117+ cells within the infarct zone four weeks following transplantation of cardiac fibroblasts stably transfected with or without SDF-1 expression vector with or without G-CSF administration for five days following cardiac fibroblast transplantation. Data represent mean±s.d. of positive cells quantified by two independent observers blinded to the identity of each animal. n=3-5 per group.

FIG. 5c is a photograph from a SDF-1/G-CSF treated animal stained CD117+. Scale bar represents 25 μM.

Consistent with SDF-1 being sufficient to induce stem cell homing to injured myocardium, in response to G-CSF, hearts transplanted with SDF-1-expressing cardiac fibroblasts revealed a greater than 3-fold increase in the number of BrdU-positive cells throughout the infarct zone. Animals that received control cardiac fibroblasts had a BrdU signal that was no different from control.

Identification of BrdU Positive Cells

We performed immunofluorescence in order to determine the identity of the BrdU cells within the infarct zone. Antibody staining for CD45 demonstrated that <5% of the BrdU-positive cells in response to cell transplantation and G-CSF administration are leukocytes, respectively. No cardiac myosin-BrdU positive cells were observed within the infarct zone of the animals treated with G-CSF without or with SKMB or cardiac fibroblast transplantation.

Effect of VEGF Expressing SKMB and G-CSF on Neovascularization and LV Function Late following MI

Despite an increased number of BrdU-positive cells in the animals that received combined SKMB transplantation and bone marrow stimulation with G-CSF, an increase in the vascular density or increase in the number of cardiac myocytes within the infarct zone was not observed. Therefore, we studied the added effect of VEGF over-expression on SKMB transplantation and G-CSF administration.

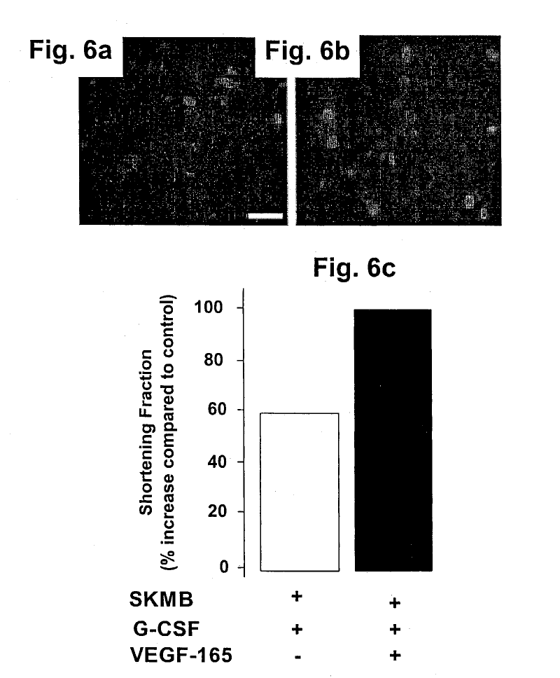

FIGS. 6(a and b) show that the immunohistochemistry of the infarct zone revealed both BrdU+ cells (open arrows) and cardiac myosin-expressing cells (closed arrows) 12 weeks following LAD ligation with cell transplantation of (a) SKMB or (b) VEGF-expressing SKMB followed by stem cell mobilization using G-CSF.

FIG. 6c shows improvement in LV function relative to no treatment control. Data represent mean±s.d. n=6-8 per group. Scale bar represents 10 μM.

Transplantation of VEGF-165-expressing SKMB 8 weeks following MI resulted in an increased vascular density within the infarct zone compared to saline controls (44.1±5.2 vs. 17.7±2.8 vessels/mm²; VEGF-165 vs. saline, respectively). Furthermore, the combination VEGF-165 expression and stem cell mobilization with G-CSF led to repopulation of the infarct zone with cardiac myosin-expressing cells consistent with myocardial regeneration (FIG. 6a). The addition of VEGF-165 expression to SKMB also significantly increased LV function as measured by shortening fraction (FIG. 6b). No significant difference was observed in shortening fraction between treatment strategies of transplantation of VEGF-165 expressing SKMB and SKMB transplantation combined with the administration of G-CSF (data not shown).

Methods

LAD Ligation

All animal protocols were approved by the Animal Research Committee, and all animals were housed in the AAALAC animal facility of the Cleveland Clinic Foundation. Animals were anesthetized with sodium pentobarbital, 50 mg/kg, intubated, and ventilated with room air at 80 breaths per minute using a pressure cycled rodent ventilator (Kent Scientific Corp, RSP1002). Anterior wall MI was induced in 150-175 g male Lewis rats by ligation of the left anterior descending (LAD) artery with the aid of a surgical microscope (Leica M500).

2D-Echocardiography

2D-echocardiography was performed 5-7 days and 8 weeks following LAD ligation and 4 weeks following SKMB transplantation using a 15-MHz linear array transducer interfaced with a Sequoia C256 (Acuson). Rats were lightly sedated with ketamine (50 mg/kg) for each echocardiogram. For quantification of LV dimensions and wall thickness, we digitally recorded 2D clips and m-mode images in a short axis view from the mid-LV just below the papillary muscles allowing for consistent measurements from the same anatomical location in different rats. Measurements were made by two independent blinded observers offline using ProSolve. Each measurement in each animal is made six times, from three randomly chosen m-mode clips out of five recorded by an observer blinded to treatment arm. As a measure of LV function, the shortening fraction was calculated from M-Mode recordings. Shortening fraction (%)=(LVEDD-LVESD)/LVEDD*100, where LVEDD—left ventricular end diastolic dimension and LVESD—left ventricular end systolic dimension. Dimensions were measured between the anterior wall and posterior wall from the short axis view just below the level of the papillary muscle. In addition, anterior wall thickness was measured at end-diastole.

Cell Preparation and Delivery

Skeletal myoblasts were harvested from the hind limbs of several Lewis rats (Harlan Labs), plated in 175 ml culture flask (Falcon), and grown in DMEM including 10% fetal bovine serum, 300 mg/l ECGS, and the antibiotics penicillin, streptomycin, and ofloxacin. Cells underwent passaging once 75% confluence was achieved to avoid differentiation. On the day prior to cell transplantation, purified myoblasts were transfected with 108 pfu/ml of replicative deficient adenovirus expressing VEGF-165 or luciferase (control) under control of a CMV promoter. On the day of transplantation, myoblasts were harvested with trypsin, washed extensively in PBS to remove any free viral particles and reconstituted immediately prior to transplantation. Animals were then anesthetized, ventilated and subjected to a lateral thoracotomy for direct visualization of the infarct zone. Approximately 1×10⁶ cells were injected per animal in five locations.

Cardiac fibroblasts were harvested from several adult rat hearts and plated in a similar fashion to SKMB. SDF-1 from the total RNA retrieved from hearts 24 h after myocardial infarction was cloned into the expression vector PCDNA3.1

(Forward-(NOT-1)-
AATAAGAAATGCGGCCGCATGGACGCCAAGGTCGTCGCTGTGCTGGCC;
Reverse-(Xba-1)-
TCTAGACTTGTTTAAGGCTTTGTCCAGGTACTCTTGGA.

Cardiac fibroblasts stably transfected with SDF-1 PCDNA3.1 expression vector were selected with neomycin.

Stem Cell Mobilization

Recombinant human G-CSF (125 ug/kg) was administered via intraperitoneal (i.p.) injection for five days beginning on the day of skeletal myoblast transplantation. Complete blood count and differential data (Bayer, ADVIA) were obtained on day 0, 5, 14, and 21 post transplantation. In order to measure the cumulative extent of cell proliferation, 50 mg/kg of BrdU was injected i.p. for 14 days beginning on day 5 to allow for BrdU labeling of any proliferating stem cells induced by G-CSF.

Histologic Analysis

Rats were euthanized, their hearts harvested for analysis four weeks following cell transplantation following perfusion fixation with HistoChoice (Amresco Inc., Solon, Ohio), and sectioned into three equal division perpendicular to the LV long-axis. The mid-ventricular and apical segments were paraffin-embedded and several sections 6 um thick were utilized for immunohistochemistry. Monoclonal antibodies to cardiac myosin (Chemicon), CD45 (Chemicon), Factor VIII (Chemicon), CD117 (Santa Cruz Biotehnology) and BrdU (Vector labs) were utilized. Secondary antibodies either FITC- or biotin-labeled were used. For quantification, five sections within the infarct zone were analyzed for positive cells and vascular density. We were unable to perform CD117 and BrdU double labeling because the HCl treatment in our protocol for BrdU antigen presentation resulted in the expression of a nuclear epitope that three different CD117 antibodies recognized.

PCR Analysis

RT-PCR analysis was performed on total RNA isolated from rat hearts as a function of time after LAD ligation and after SKMB transplantation. Total RNA was extracted from tissue by the guanidine isothiocyanate-cesium chloride method. Primers specific for rat SDF-1 (Forward: TTGCCAGCACAAAGACACTCC; Reverse: CTCCAAAGCAAACCGAA TACAG, expected product 243 base pairs, 40 cycles) were utilized and GAPDH (Forward: CCCCTGGCCAAGGTCATCCA; Reverse: CGGAAGGCCATGCCAGTGAG, expected product 238 base pairs, 20 cycles). Real time PCR (Perkin-Elmer, ABI Prism 7700) was then used to confirm the increase in expression within infarcted and transplanted hearts using SYBR-green incorporation into the PCR product SDF-1 (Forward: ATGCCCCTGCCGATTCTTTG Reverse: TGTTGTTGCTTTTCAGCCTTGC, expected product 116 base pairs) and GAPDH as above.

Adenoviral Construct

The adenoviral construct encoding VEGF-165 was generous gift from Gen Vec, Inc (Gaithersburg, Md.). Briefly, 293 cells were obtained from American Type Culture Collection (ATCC CRL. 1573) and were maintained in Dulbecco's modified Eagle's medium (DMEM) supplemented with 10% calf serum. The E1-,E3-adenovirus vector AdVEGF-165 was generated by linearizing the shuttle vector plasmid at a unique restriction site adjacent to the left end inverted terminal repeat (ITR) and cotransfected into 293 cells with ClaI digested H5d1324 DNA. After two sequential plaque purifications vector stocks were propagated on 293 cells and purified through three sequential bandings on cesium chloride gradients. The purified virus was dialyzed against a buffer containing 10 mM Tris, pH 7.8, 150 mM NaCl, 10 mM MgCl2 and 3% sucrose and stored at −80 0C until use. The transgene expression is under the control of the cytomegalovirus immediate early promoter.

Statistical Analysis

Data are presented as mean±SEM. Comparisons between groups were made by Student t test.

Second Series of Examples

Effect of Skeletal Myoblast Transplantation on Left Ventricular Function

The effects of autologous SKMB transplantation on vascular density and LV function were examined to determine if SKMB expressing VEGF-165 leads to significant neovascularization of the infarct zone and improved left ventricular function.

Eight weeks following myocardial infarction induced by LAD ligation, the peri-infarct zone was injected with either 1 million SKMB (n=6) or saline (n=7) in five equally divided injections. Four weeks later LV function was quantified as shortening fraction by echocardiogram, and the hearts were harvested. The vasculature was identified by Factor VIII immunohistochemistry and vascular density was quantified throughout the infarct zone.

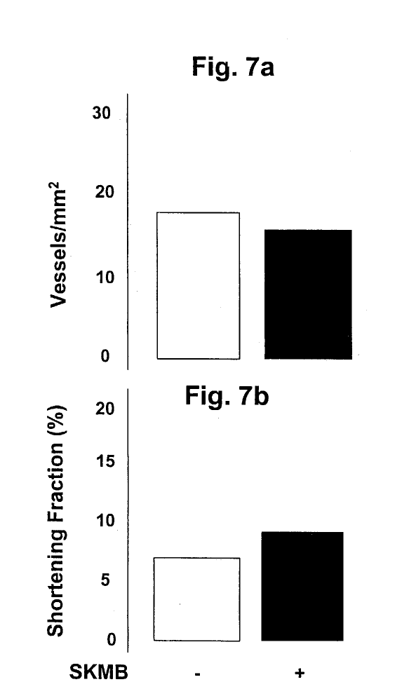

FIGS. 7(a and b) graphically illustrates the data from this study. Data represent mean±sd, *P<0.01.

The data shows that the transplantation of SKMB into the peri-infarct zone eight weeks after myocardial infarction induced by LAD ligation did not result in neovascularization of the infarct zone (7a). SKMB transplantation, however, did result in a small (˜20%), yet statistically significant increase in LV function as measured by shortening fraction at the level of the papillary muscle (6.8±1.0% vs. 8.1±1.1%, P<0.01) (7b).

Neovascularization in Response to Cell-based and Direct Adenoviral VEGF Delivery

The angiogenic response between direct viral injections was compared to transplantation of virally transfected SKMB. In each case, eight weeks following myocardial infarction induced by LAD ligation, the peri-infarct zone was injected with either 1×10⁷ pfu of AdVEGF-165 (n=4) or 1 million SKMB transfected with AdLuc (n=3) or AdVEGF-165 (n-6) in five equally divided injections. The vasculature was identified by Factor VIII immunohistochemistry and vascular density was quantified by counting Factor VIII stained vessels in tissue sections obtained just below the level of the papillary muscles.

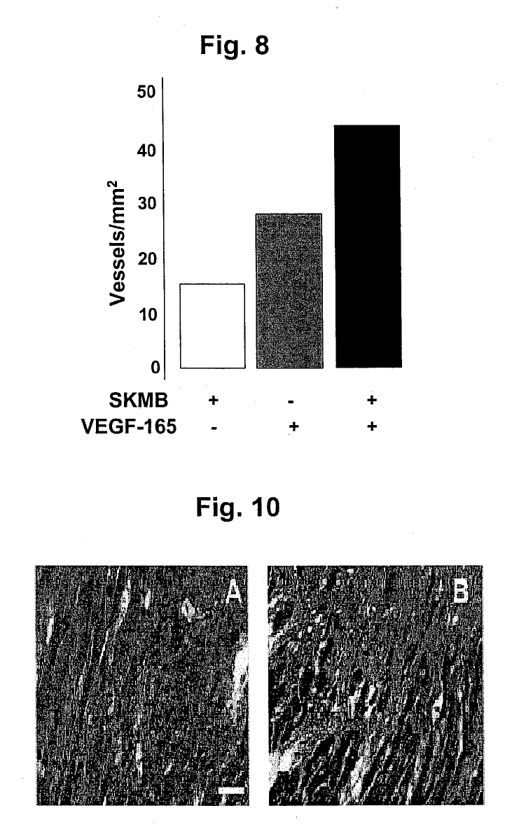

FIG. 8 shows the vessel density within the infarct zone by direct adenoviral injection compared to the vessel density by transplantation of cells expressing VEGF-165. Data represent mean±sd, *P<0.05.

A significant increase in vascular density was observed in those animals that received either adenoviral injection of VEGF-165 or cell transplantation with VEGF-165 expressing SKMB. There was no gross or histologic evidence of hemangioma formation with either treatment strategy. A significant greater increase in vascular density in those animals treated with cell transplantation was also observed compared to direct viral injection.

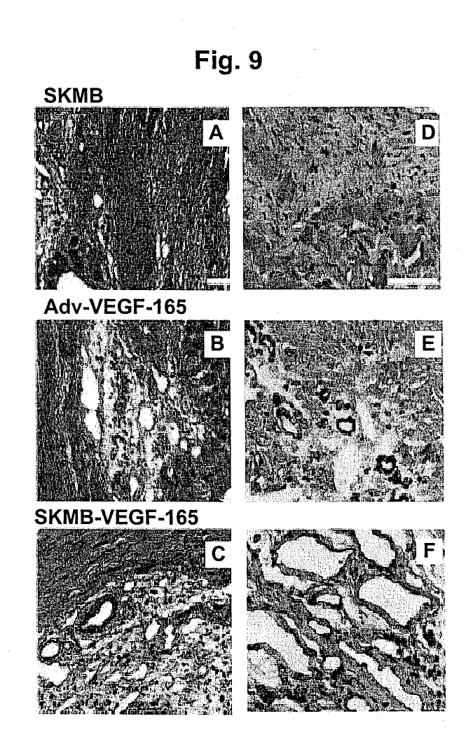

FIGS. 9(a-f) are photographs of representative sections of the infarct zone 4 weeks following injection into the peri-infarct zone of either 1 million SKMB transfected with AdLUC (A, D), 1×10⁷ pfu AdVEGF-165 (B, E) or 1 million SKMB transfected with AdVEGF-165 (C, F) in five equally divided injections. (A-C)H & E staining and (D-F) immunohistochemistry for Factor VIII.

The photographs in FIGS. 9(a-f) show that the infarct zone following SKMB transplantation is relatively avascular (FIGS. 9(a and d)). The neovascularization following VEGF-165 therapy by either modality results in the development of increased vascular density was characterized by an increase in the number of capillaries and small arterioles (FIGS. 9(b, c, e, and f)).

FIGS. 10(a and b) show representative H & E stained sections of the peri-infarct zone four weeks following injection of (a) 1×10⁷ pfu AdVEGF-165 or (b) 1 million SKMB transfected with 1×10⁷ pfu AdVEGF-165 each in five equally divided injections.

Four weeks following treatment, the peri-infarct zone in animals injected with adenovirus consistently revealed an inflammatory infiltrate (FIG. 10a) that was not present in any of the animals transplanted with VEGF-165 expressing SKMB (FIG. 10b).

Cell-Based Delivery of VEGF Results in Improved Left Ventricular Function

To determine if either direct viral injection or cell-based expression of VEGF-165 leads to improved LV function, eight weeks following myocardial infarction the peri-infarct zone was injected with either saline, 1×10⁷ pfu of AdVEGF-165 (n=4) or 1 million SKMB transfected with AdLuc (n=3) or AdVEGF-165 (n=6) in five equally divided injections. LV function was quantified 4 weeks later by echocardiogram.

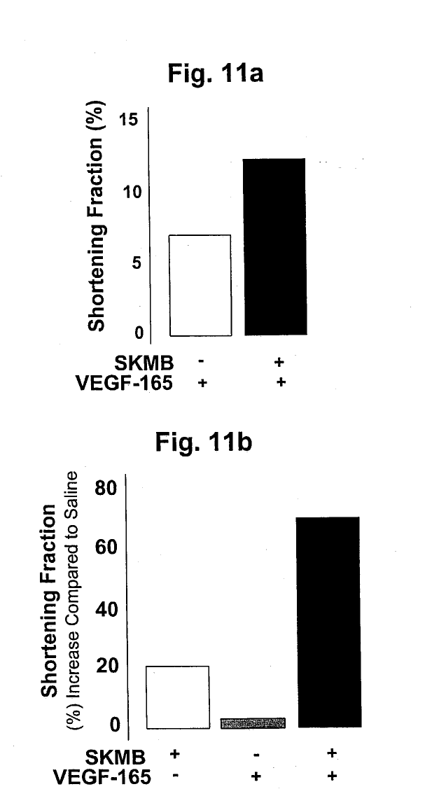

FIGS. 11 (a and b) show LV function presented as (A) shortening fraction (%) or (B) relative to saline control. Data represent mean±sd, *P<0.01.

The LAD ligation model used for these studies resulted in a significant decrease in shortening fraction. There was a significant increase in LV function in the hearts that underwent transplantation with cells expressing VEGF-165 compared to hearts that received direct injection of adenovirus encoding for VEGF-165 (FIG. 11a). Furthermore, the improvement in LV function seen with transplantation of VEGF-165 expressing SKMB was significantly greater than that seen with transplantation of SKMB alone (FIG. 11b). Despite a significant increase in vascular density with direct injection of VEGF-165 encoding adenovirus, no improvement in LV function compared to saline injection alone was seen with this treatment strategy (FIG. 11b).

The data from these second series of experiments demonstrate that both adenoviral and cell-based delivery of VEGF-165 induces neovascularization (50±7% and 145±29% increase in vascular density compared to SKMB alone, respectively), within the infarct zone. Cell-based, but not adenoviral delivery of VEGF-165, resulted in a significant increase in cardiac function (69.1±8.2% and 1.5±5.8% increase in shortening fraction compared to saline control), even when compared to the delivery of SKMB alone (increase of 19.1±10.7%). The data from these second series of experiments further demonstrate that cell-based delivery of VEGF leads to an improved treatment-effect over direct adenoviral injection, and suggest that already developed adenoviral vectors could potentially be used as adjunctive therapy when considering SKMB transplantation.

Both of these approaches resulted in the local transient expression of VEGF, and all animals in the study were ultimately treated with 1×10⁷ pfu of VEGF encoding adenovirus.

Significant neovascularization was seen throughout the infarct zone with both VEGF delivery strategies, and a small increase in left ventricular function was observed in those animals treated with SKMB alone. The transplantation of VEGF-165 expressing SKMB resulted in a significantly greater vascular density and ˜70% increase in LV function, as quantified by shortening fraction. On the other hand, despite an increase in vascular density within the infarct zone with the injection of adenovirus encoding VEGF-165, this therapy did not result in an improvement in LV function.

Potential mechanisms for the synergistic improvement in LV function observed using concurrent SKMB and VEGF may relate to the ability of VEGF to induce hematopoietic stem cell (HSC) release from the bone marrow. Vascular endothelial growth factor (VEGF) administration has been shown to mobilize CD34+ hematopoietic stem cells in mice, resulting in augmented neovascularization. It is possible that both delivery strategies similar HSC release, but that in the absence of the inflammatory response typically associated with adenoviral injection (and not associated with transplantation of VEGF-165 expressing SKMB), the HSC that enter the myocardium may be more likely to differentiate into cardiac myocytes.

Methods

LAD Ligation

All animal protocols were approved by the Animal Research Committee, and all animals were housed in the AAALAC animal facility of the Cleveland Clinic Foundation. Animals were anesthetized with sodium pentobarbital, 50 mg/kg, intubated, and ventilated with room air at 80 breaths per minute using a pressure cycled rodent ventilator (Kent Scientific Corp, RSP1002). Anterior wall MI was induced in 150-175 g male Lewis rats by ligation of the left anterior descending (LAD) artery with the aid of a surgical microscope (Leica M500).

2D-Echocardiography

2D-echocardiography was performed 5-7 days and 8 weeks following LAD ligation and 4 weeks following SKMB transplantation using a 15-MHz linear array transducer interfaced with a Sequoia C256 (Acuson). Rats were lightly sedated with ketamine (50 mg/kg) for each echocardiogram. For quantification of LV dimensions and wall thickness, we digitally recorded 2D clips and m-mode images in a short axis view from the mid-LV just below the papillary muscles allowing for consistent measurements from the same anatomical location in different rats. Measurements were made by two independent blinded observers offline using ProSolve. Each measurement in each animal is made 6 times, from 3 randomly chosen m-mode clips out of 5 recorded by an observer blinded to treatment arm. As a measure of LV function, the shortening fraction was calculated from M-Mode recordings. Shortening fraction (%)=(LVEDD-LVESD)/LVEDD*100, where LVEDD—left ventricular end diastolic dimension and LVESD—left ventricular end systolic dimension. Dimensions were measured between the anterior wall and posterior wall from the short axis view just below the level of the papillary muscle. In addition, anterior wall thickness was measured at end-diastole.

Cell Preparation and Cell and Viral Delivery:

Skeletal myoblasts were harvested from the hind limbs of several Lewis rats (Harlan Labs), plated in 175 ml culture flask (Falcon), and grown in DMEM including 10% fetal bovine serum, 300 mg/l ECGS, and the antibiotics penicillin, streptomycin, and ofloxacin. Cells were passed once 75% confluence was achieved to avoid differentiation.

On the day prior to cell transplantation, purified myoblasts were transfected with 1×10⁷ pfu/ml of replication deficient, E1, E3-deleted adenovirus expressing VEGF-165 or luciferase (control) both under control of a CMV promoter. On the day of transplantation, myoblasts were harvested with trypsin, washed extensively in PBS to remove any free viral particles and reconstituted immediately prior to transplantation. Animals were then anesthetized, ventilated and subjected to a lateral thoracotomy for direct visualization of the infarct zone. Approximately 1×10⁶ cells were injected per animal in five locations. Similarly, direct viral injection into the peri-infarct was accomplished through five injections of 0.2×10⁷ pfu each. Volume of each injection was 100 μL. In all experiments, two injections were made along the left and two along the right border of the peri-infarct zone; the fifth injection was in the peri-infarct zone at the LV apex.

Adenoviral Construct

The adenoviral construct encoding VEGF-165 was generous gift from Gen Vec, Inc (Gaithersburg, Md.). Briefly, 293 cells were obtained from American Type Culture Collection (ATCC CRL. 1573) and were maintained in Dulbecco's modified Eagle's medium (DMEM) supplemented with 10% calf serum. The E1-,E3-deleted adenovirus vector AdVEGF-165 was generated by linearizing the shuttle vector plasmid at a unique restriction site adjacent to the left end inverted terminal repeat (ITR) and cotransfected into 293 cells with ClaI digested H5d1324 DNA. After two sequential plaque purifications vector stocks were propagated on 293 cells and purified through three sequential bandings on cesium chloride gradients. The purified virus was dialyzed against a buffer containing 10 mM Tris, pH 7.8, 150 mM NaCl, 10 mM MgCl2 and 3% sucrose and stored at −80 0C until use. The transgene expression is under the control of the cytomegalovirus immediate early promoter.

Histologic Analysis

Rats were euthanized, their hearts harvested for analysis 4 weeks following cell transplantation following perfusion fixation with HistoChoice (Amresco Inc., Solon, Ohio), and sectioned into three equal divisions perpendicular to the LV long-axis. The mid-ventricular section was paraffin-embedded and several sections 6 um thick were obtained just below the papillary muscle for analysis. Sections were stained with hemotoxylyn and eosin for histological analysis. To assist with blood vessel identification, sections were stained using an antibody to Factor VIII (Santa Cruz Biotechnology) and an HRP labeled goat anti-mouse secondary antibody. These sections were counterstained with hematoxylin. Blood vessels were counted throughout the infarct zone of each animal by a trained observer blinded to the identity of each animal.

Statistical Analysis

Data are presented as mean±s.d. Comparisons between groups were made by Student t-test.

Having described the invention, the following is claimed:

1. A method of stimulating stem cell differentiation in ischemia damaged tissue, said ischemia damaged tissue including a first concentration of stem cells and a first concentration of VEGF, said method comprising the steps of:
increasing the concentration of VEGF in said ischemia damaged tissue from the first concentration to a second concentration; and
increasing the concentration of stem cells in said ischemia damaged tissue from said first concentration to a second concentration, said concentration of stem cells being increased while said concentration of VEGF in said ischemia damaged tissue is increased.

2. The method of claim 1 wherein the step of increasing the concentration of stem cells comprises administering an agent that causes the stem cells to mobilize from bone marrow to peripheral blood of the ischemia damaged tissue.

3. The method of claim 2 wherein the agent that causes the stem cells to mobilize from the bone marrow to the peripheral blood is selected from the group consisting of cytokines, chemokines, and the chemotherapeutic agents.

4. The method of claim 2 wherein the agent comprises G-CSF.

5. The method of claim 1 wherein the step of increasing the number of stem cells comprises injecting stem cells into the peripheral blood.

6. The method of claim 1 wherein the step of increasing the concentration of VEGF comprises affecting cells to express VEGF in the ischemia damaged tissue.

7. The method of claim 6 wherein the cells are affected to express VEGF using gene therapy.

8. The method of claim 6 wherein the cells affected to express VEGF comprise cells that have been cultured ex vivo and introduced into the ischemia damaged tissue.

9. The method of claim 8 wherein the cells affected to express VEGF comprise autologous cells that have been harvested from the subject to be treated prior to culturing.

10. The method of claim 6 wherein the cells affected to express VEGF comprise cells of the ischemia damaged tissue.

11. The method of claim 6 wherein the ischemia damaged tissue comprises a first concentration of SDF-1, and further comprising the step of increasing the concentration of SDF-1 in the ischemia damaged tissue from the first concentration to a second concentration while the concentration of VEGF in the ischemia damaged tissue is increased.

12. The method of claim 11 wherein the concentration of SDF-1 in the ischemia damaged tissue is increased by affecting cells in the ischemia damaged tissue to express SDF-1.

13. The method of claim 11 wherein the cells are affected to express SDF-1 by introducing an expression vector into the cells, said expression vector including a nucleic acid encoding for SDF-1.

14. The method of claim 13 wherein the cells affected to express SDF-1 comprise cells that have been cultured ex vivo and introduced into the ischemia damaged tissue.

15. The method of claim 14 wherein the cells affected to express SDF-1 comprise autologous cells that have been harvested from the subject to be treated prior to culturing.

16. The method of claim 6 wherein the cells affected to express SDF-1 comprise native cells of the ischemia damaged tissue.

17. A method of stimulating stem cell differentiation in infarcted myocardium, said method comprising the steps of:
introducing cells into said infarcted myocardium, said cells being affected to express VEGF in said infarcted myocardium; and
administering an agent that mobilizes said stem cells from bone marrow to peripheral blood of the infarcted myocardium, said stem cells being mobilized from the bone marrow to the peripheral blood while said VEGF is expressed in the infarcted myocardium.

18. The method of claim 17 wherein the agent that causes the stem cells to mobilize from the bone marrow to the peripheral blood is selected from the group consisting of cytokines, chemokines, and the chemotherapeutic agents.

19. The method of claim 18 wherein the agent comprises G-CSF.

20. The method of claim 17 wherein the cells are affected to express VEGF using gene therapy.

21. The method of claim 20 wherein the cells are affected to express VEGF, are transfected by an expression vector, said expression vector including a nucleotide encoding VEGF.

22. The method of claim 17 further comprising the step of culturing cells ex vivo prior to affecting the cells to express VEGF.

23. The method of claim 22 wherein the cells affected to express VEGF comprise autologous cells that have been harvested from the subject to be treated prior to culturing.

24. The method of claim 17 wherein said infarcted myocardium includes a first concentration of VEGF, the cells affected to express VEGF increasing the concentration of VEGF in the ischemia damaged tissue from the first concentration to a second concentration.

25. The method of claim 17 wherein the cells affected to express VEGF in the infarcted myocardium being further affected to express SDF-1 in the ischemia damaged tissue.

26. The method of claim 17 wherein the cells are affected to express SDF-1 by introducing an expression vector into said cells ex vivo, said expression vector including a nucleic acid encoding for SDF-1.

27. A method of stimulating stem cell differentiation in infarcted myocardium, said method comprising the steps of:
introducing skeletal myoblasts into said infarcted myocardium, said skeletal myoblasts being transfected with an expression vector to express VEGF in said infarcted myocardium; and
administering a colony stimulating factor that mobilizes said stem cells from bone marrow to peripheral blood of the infarcted myocardium, said stem cells being mobilized from the bone marrow to the peripheral blood while said VEGF is expressed in the infarcted myocardium.

28. The method of claim 27 wherein said stem cells mobilized from the bone marrow are differentiated into cardiomyocytes.

29. The method of claim 27, wherein said stem cell differentiation promotes tissue regeneration in the infarcted myocardium.


SEQUENCE LISTING (8 sequences)

SEQ ID NO: 1 - DNA, 48 bp, Artificial
  vector
aataagaaatgcggccgcatggacgccaaggtcgtcgctgtgctggcc

SEQ ID NO: 2 - DNA, 38 bp, Artificial
  PCDNA3.1 vector
tctagacttgtttaaggctttgtccaggtactcttgga

SEQ ID NO: 3 - DNA, 21 bp, Artificial
  oligonucleotide primer
ttgccagcacaaagacactcc

SEQ ID NO: 4 - DNA, 22 bp, Artificial
  oligonucleotide primer
ctccaaagcaaaccgaatacag

SEQ ID NO: 5 - DNA, 20 bp, Artificial
  oligonucleotide primer
cccctggccaaggtcatcca

SEQ ID NO: 6 - DNA, 20 bp, Artificial
  oligonucleotide primer
cggaaggccatgccagtgag

SEQ ID NO: 7 - DNA, 20 bp, Artificial
  oligonucleotide primer
atgcccctgccgattctttg

SEQ ID NO: 8 - DNA, 22 bp, Artificial
  oligonucleotide primer
tgttgttgcttttcagccttgc